(19) United States
(12) Patent Application Publication
(10) Pub. No.: US 2012/0316451 A1
(43) Pub. Date: Dec. 13, 2012

(54) Event Evaluation Using Heart Rate Variation for Ingestion Monitoring and Therapy

(75) Inventors: Rose Province, San Jose, CA (US); Mike Hedman, Saratoga, CA (US); Matthew Hills, Los Altos, CA (US); John C. Potosky, San Jose, CA (US)

(73) Assignee: IntraPace, Inc., Mountain View, CA (US)

(21) Appl. No.: 13/315,091

(22) Filed: Dec. 8, 2011

Related U.S. Application Data
(60) Provisional application No. 61/421,150, filed on Dec. 8, 2010.

(51) Int. Cl. A61N 1/36; A61B 5/00; A61B 5/024

(52) U.S. Cl. 600/508; 600/300; 607/40

(57) ABSTRACT

Medical, diagnostic, and/or patient monitoring methods, systems and devices enhance ingestion-related health, often by screening and/or treating patients with eating disorders. Optionally, a gastric electric stimulation (GES) therapy system monitors changes in an obese patients' autonomic balance associated with a stimulation event and/or a meal event by analyzing heart rate variability (HRV) of the patient. These event-based changes in autonomic balance may be used to determine which patients will likely respond to GES therapy, and/or to control the GES therapy administration.

### CROSS-REFERENCES TO RELATED APPLICATIONS

The present application claims the benefit under 35 USC 119(e) of U.S. Provisional Application No. 61/421,150 filed Dec. 8, 2010. The full disclosure of which is incorporated herein by reference in its entirety for all purposes.

The subject matter of the present application is related to that of the following applications: U.S. patent application Ser. No. 12/145,430 filed on Jun. 24, 2008 (our Ref. No. 026458-000610US), U.S. patent application Ser. No. 10/950,345 filed on Sep. 23, 2004 (our Ref. No. 026458-000141US), U.S. patent application Ser. No. 12/637,452 filed on Dec. 14, 2009 (our Ref. No. 026458-001110US), and U.S. patent application Ser. No. 12/754,435 filed on Apr. 5, 2010 (our Ref. No. 026458-001210US), all of which are herein incorporated by reference.

### BACKGROUND OF THE INVENTION

1. Field of Invention

The present invention relates generally to medical methods, systems and devices, and/or to the monitoring of ingestion and ingestion-related health. More particularly, embodiments of the present invention relate to screening and treating obese patients, patients with an eating disorder, and the like.

Since the mid-seventies, the prevalence of obesity has increased sharply for both adults and children. These increasing rates raise concern because of their implications for Americans' health. Being overweight or obese may increase the risk of many diseases and health conditions, including: hypertension, dyslipidemia (for example, high total cholesterol or high levels of triglycerides), type 2 diabetes, coronary heart disease, stroke, gallbladder disease, osteoarthritis, sleep apnea and respiratory problems, and some cancers (such as endometrial, breast, and colon).

Obesity and its associated health problems have a significant economic impact on the U.S. health care system. Medical costs associated with excess weight and obesity may involve direct and indirect costs. Direct medical costs may include preventive, diagnostic, and treatment services related to obesity. Indirect costs relate to morbidity and mortality costs. Morbidity costs are defined as the value of income lost from decreased productivity, restricted activity, absenteeism, and bed days. Mortality costs are the value of future income lost by premature death.

Many therapies are currently being investigated for treatment of obesity and diseases associated with obesity. To date, the widely used obesity treatments have not been shown to be ideal, particularly for those afflicted with severe obesity. The approaches that have been proposed range from lifestyle coaching to major surgical therapies. Unfortunately, patient compliance and the accuracy with which patients report their own activities can significantly limit the effectiveness of coaching and support groups. Even approaches which increase the overall health of a morbidly obese patient (and which, if continued for a sufficient number of days, weeks, or even months would eventually result in major weight loss) may not be sustained, because the lack of near-term weight loss may discourage the patient. While surgical approaches can limit the capacity of the patient's food intake over a set amount of time regardless of compliance, quite severe surgical modifications may have to be imposed to achieve the desired result. Notwithstanding that, as a group, obese patients may be highly motivated to find a solution to help them lose weight and improve their health, obese individuals will often exhibit behavior which circumvents or limits the efficacy of therapies so that effective surgical approaches may have to significantly restrict gastrointestinal function, while more moderate approaches may not achieve the desired results. Nonetheless, improved awareness of obesity's role in increasing the incidence of other serious health issues is contributing to overweight consumers' desire to take a more active role in the management of their weight, lifestyle and health.

Surgical interventions have been proposed and applied that may involve less drastic (and potentially less permanent) modifications to the gastrointestinal tract. Gastric electric stimulation (GES) therapy has been used on a number of obese patients. The ultimate success and results of the GES therapy are highly dependent on individual patients' responses. What is effective for one patient, may not work for another. Additional developments of these potentially advantageous systems may enhance their overall efficacy and the number of morbidly patients that are able to see the potential benefits of significant, long-lasting weight loss.

Therefore, it would be desirable to provide devices, systems and methods that can help screen patients who would be most likely to benefit from GES therapy for obesity and obesity-related eating disorders. It would be desirable to provide devices, systems and methods that can gage the effectiveness of exploratory and/or ongoing therapeutic GES therapy on individual patients suffering from obesity or eating disorders. In light of the challenges of accurately assessing therapies that may not result in significant loss in weight, devices, systems and methods that are not fully dependent on patient compliance and self-reporting of caloric intake would provide a clearer, more objective picture of the effectiveness of GES therapy on the patient's ingestion of food. It would also be desirable to provide improved titration of the GES therapy for an individual patient so that the therapy can be adjusted and tailored for maximum efficacy, either automatically in the device, or an algorithm in an external instrument used to program the device. In addition, it would be beneficial to provide improved health diagnostics regarding the patients' autonomic nervous system balance or autonomic tone, optionally to the patient's health-care professional, from the system; this information could be used to monitor the patient's progress in addition to presenting behavior-based information to the patient for effective behavior modification and greater success in achieving weight loss or health goals.

### BRIEF SUMMARY OF THE INVENTION

The present invention generally provides improved medical, diagnostic, and/or patient monitoring methods, systems and devices, with many embodiments being particularly well suited to enhancing ingestion-related health. Some exemplary embodiments of the present invention relate to tools for screening and treating patients with eating disorders, with these tools optionally being compatible with (and/or incorporated into) a gastric electric stimulation (GES) therapy system. Unfortunately, obese patients vary in their response to GES therapy, and in their autonomic response to a meal. Advantageously, changes in an obese patients' autonomic balance associated with stimulation and/or a meal may be identified and monitored using heart signals. Event-based changes in autonomic balance information may be used to determine which patients will likely respond to GES therapy, and/or to control the GES therapy administration. Rather than (or possibly in addition to) merely taking daily measurements of autonomic balance and imposing long-term continuous stimulation with the goal of seeking to gradually alter resting autonomic balance toward a healthy value, the systems described herein will often monitor a patient at least intermittently during a day, and may also provide an evaluation of intra-day variations in autonomic balance values associated with eating of a meal (or other ingestion), stimulation applied to tissues of (or nerves associated with) a gastrointestinal tract of the patient, or the like. Embodiments of the invention may make use of heart rate variability (HRV) or other autonomic balance indicators as a feedback signal, thereby helping to promote adoption of GES systems via improved include patient selection, enhanced stimulation site selection, improved stimulation dose titration and adjustments over time, more effective stimulation timing control, lower overall system cost and complexity, and/or the like.

In a first aspect, the invention provide a system for performing diagnostic or therapeutic functions for a patient having an ingestion-related disorder. The system comprises a sensor configured to collect data from the patient, and a treatment applicator or display. A processor couples the sensor to the treatment applicator or display. The processor is configured to identify a baseline autonomic nervous system balance in response to the sensed data. The processor is also configured to identify an excursion of the autonomic nervous system balance from the baseline, the excursion associated with a discrete ingestion or stimulation event; evaluate the event using the baseline autonomic system balance; and to transmit command signals to the treatment applicator or display in response to the evaluation of the event so as to promote ingestion modification by the patient.

In many embodiments, the sensor comprises a heart beat signal sensor and the autonomic nervous system balance baseline and excursion are determined by generating heart rate variability information before, during, and/or after the event. The heart beat signal sensor can be configured to be implanted into a body of the patient, and the treatment applicator may be configured to be implanted into the body coupled to a tissue of the gastrointestinal tract or associated nerves of the patient to stimulate the tissue so as to inhibit unhealthy ingestion into the patient. The stimulation will often be applied by the processor in response to the heart rate variability information. The treatment applicator, when implanted, may stimulate the tissue in response to the command signals during the event, particularly when the event includes ingestion into the patient body. The processor can be configured to identify the event as an unhealthy ingestion event using at least the heart rate variability information.

In some embodiments, the event may comprise a stimulation event. The processor can be configured to alter stimulation applied by the treatment applicator in response to the heart rate variability information, and the evaluation of the event may include an evaluation of effectiveness of the stimulation during the stimulation event. For example, the stimulation effectiveness may be evaluated by determining if the stimulation induces a desired temporary excursion from a baseline autonomic nervous system balance during and/or after the stimulation. The processor can be configured to initiate the stimulation event in response to ingestion by the patient, and the event may include both an ingestion event and a stimulation event. In some embodiments, the display may show patient selection information in response to the command signals, particularly where the stimulation applicator includes a patient evaluation probe and the stimulation event includes a patient evaluation stimulation event.

In another aspect, the invention provides a system for performing diagnostic or therapeutic functions for a patient having an ingestion-related disorder. The system comprises a sensor configured to collect data from a tissue within the patient, and a display. A processor couples the sensor to the display, and the processor can be configured to determine autonomic nervous system balance information in response to the sensed data. The processor can also be configured to transmit command signals to the display so as to generate an output. A gastric stimulator system may also be included, with the stimulation system having a stimulation surface coupleable to a tissue of a gastrointestinal tract or associated nerves in response to the output of the display.

For many embodiments, the patient will be an obese patient. The processor can be configured to receive a first set of the data associated with a first portion of a time span taken before an ingestion event while the obese patient is resting. A second set of the data may be associated with a second portion of the time span taken while the obese patient is exercising. A third set of the data may be associated with a third portion of the time span during an ingestion event of solid and/or liquid material into the obese patient. The processor can be configured to calculate an overall autonomic nervous system balance of the body for the time span, and the output by the display can indicate whether the gastric electrical stimulation therapy would be an effective treatment of obesity in the patient. Some embodiments may include an endoscopic probe having a test stimulation surface for stimulating a candidate location on a wall of a stomach of an obese patient. The output of the display may indicate whether a gastric electrical stimulation therapy with the stimulation surface at the candidate location of the lead will be effective.

In another aspect, the invention provides a method for monitoring of an individual (and optionally for treating a patient having an ingestion-related disorder). The method comprises collecting heart rate variability data from the patient, and indentifying an unhealthy ingestion event in response to the heart rate variability data. Ingestion modification by the patient can be promoted in response to the identification of the event.

In yet another aspect, the invention provides a method for selecting a patient for an ingestion-behavior modification therapy. The method comprises stimulating a tissue of the gastrointestinal tract or associated nerves of the patient. Heart rate variability data is collected from the patient while stimulation of the tissue induces a discrete change in the heart rate variability data. The patient is screened for implantation of an ingestion-behavior modification implant in response to the change in the heart rate variability data.

In one additional aspect, the invention provides a method for controlling a therapy for an obese patient. The method comprises stimulating a tissue of the gastrointestinal tract or associated nerves of the patient. Heart rate variability data is collected from the patient while stimulation of the tissue induces a discrete change in the heart rate variability data. The stimulation of the tissue is altered in response to the change in the heart rate variability data, the heart rate variability data providing a stimulation effectiveness feedback signal. Autonomic balance data is optionally collected from the patient while stimulation of the tissue induces a discrete change in the autonomic balance data. The stimulation of the tissue can be altered in response to the change in the autonomic balance data, the autonomic balance data providing a stimulation effectiveness feedback signal.

### BRIEF DESCRIPTION OF THE DRAWINGS

FIGS. 1A and 1B schematically illustrate alternative embodiments of a stimulation system and sensors of the present invention.

FIG. 2 schematically illustrates an embodiment of a treatment system of the present invention.

FIGS. 3A-3D illustrates treatment methods according to embodiments of the present invention.

FIGS. 4A-4B illustrate communication methods according to embodiments of the present invention.

FIG. 5 shows the autonomic tone during a meal for a normal versus an obese patient.

FIG. 6 shows how measurement of HRV and gastric ANS tone can be used to determine if a patient is a good candidate for GES therapy.

FIG. 7 shows how HRV can be used to detect ingestion of a meal of 400 calories or more.

FIG. 7A graphically illustrates signal changes that may be correlated with an ingestion event.

FIG. 8 shows the application of GES to normalize autonomic tone during a meal.

FIG. 9 shows a method of using HRV for GES therapy adjustment.

### DETAILED DESCRIPTION OF THE INVENTION

The methods, systems and devices described herein offer improvements over techniques currently used to screen and administer GES therapy for treatment of obesity and obesity-related eating disorders. Although some embodiments of the invention make specific reference to treatment for obesity, the methods, systems and devices described herein may be applicable to any treatment in which presenting feedback regarding patients' state of cardiac health is desired.

The human autonomic nervous system is the branch of the nervous system that controls involuntary actions such as digestion, heart rate, breathing, etc. The autonomic nervous system includes 2 branches: the sympathetic nervous system (also known as the catabolic system, and which triggers the burning of fuel to produce energy); and the parasympathetic nervous system (also known as the anabolic system). The parasympathetic nervous system includes the vagus nerve, and generally regulates processes that absorb and store energy, along with promoting the growth of muscles and tissue. The vagus nerve innervates the stomach among many other organs; upon ingestion of food, vagal activity is initially activated.

Obesity may be categorized into two types. The first type of obesity, caused by disease or hormone imbalances in the body (e.g., hypothalamic obesity) may account for only approximately 1% of the total obese population. The second type, caused by eating habits and lifestyle, may account for close to 99% of the total obese population. This latter type of obesity may be connected with the “metabolic syndrome.” The metabolic syndrome can be defined by 5 components: abdominal obesity, high triglyceride and other lipoprotein in blood, impaired insulin sensitivity, hypertension, hyperglycemia, and a systematic pro-inflammatory state. Obesity seems to be the driving force behind this syndrome; it may be present in 60% and 50% of obese men and women. Elevated sympathetic tone may be the mechanism behind the metabolic syndrome and other obesity related illnesses such as hypertension, insulin resistance, diastolic dysfunction, and renal impairment.

The sympathetic system may also be important in the generation of both obesity and obesity related illness. Acute sympathetic outflow may increase levels of fatty acids in plasma, produce more gluconeogenesis by the liver, and/or moderate inhibition of insulin release by the pancreas to conserve glucose and to shift fuel metabolism of muscle in the direction of fatty acid oxidation. If sympathetic nervous activation is sustained over a long period of time, then the next effect may be hypertension and/or development of insulin resistance.

The sympathetic nervous system may be important in virtually all of the components of daily energy expenditure including: resting metabolic rate, energy expenditure (EE) associated with physical activity, thermic effect of food, cold induced thermogenesis, and thermogenesis related to stimulants including caffeine and nicotine. The autonomic response of an obese person to stimuli such as a meal may be blunted or smaller than that of the non-obese or general population. This blunted sympathetic response could contribute to deficient thermogenesis, positive energy balance, and weight gain.

### Stimulator and Sensor System:

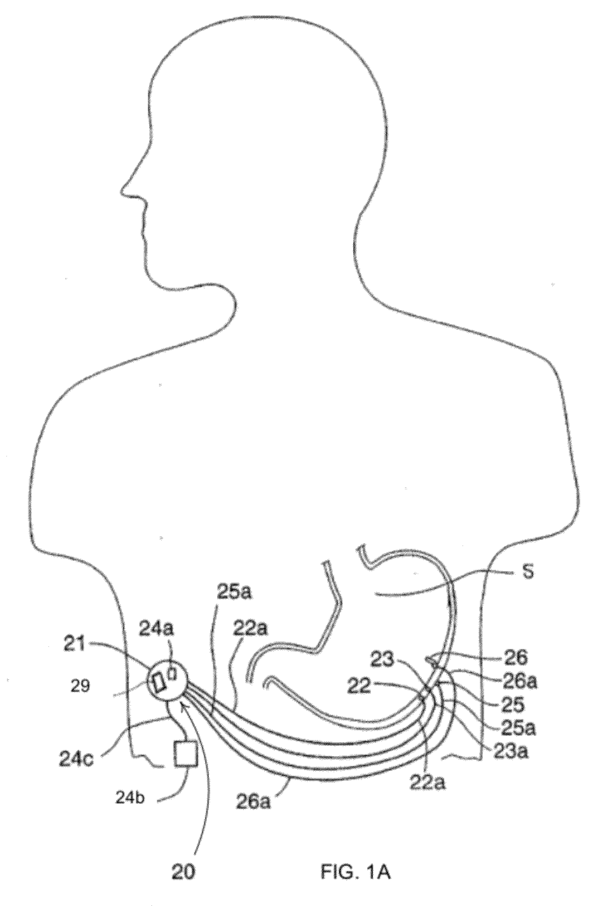

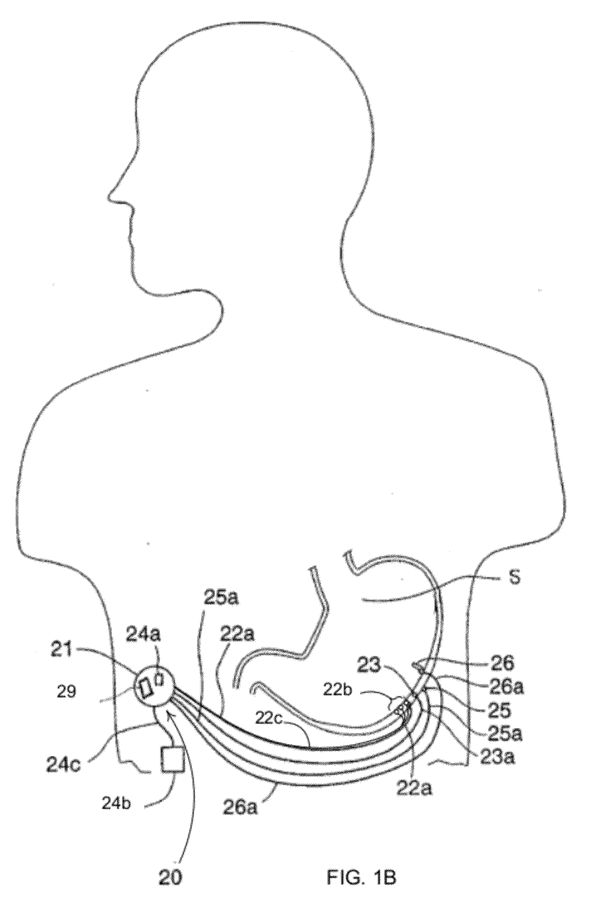

FIG. 1A schematically illustrates a system including a stimulator 20 having an implantable pulse generator (IPG) 21 or implantable device housing (CAN) implanted subcutaneously within a living body. The stimulator further comprises leads 22a and 23a extending from the IPG 21 through the abdomen and to the stomach S where electrodes 22 and 23 are implanted into the stomach muscle layer from the outside of the stomach S. The IPG 21 further comprises a sensor 24a located on the IPG 21 and/or a sensor 24b desirably separate from the IPG and located elsewhere in the patient and coupled to the electronic circuitry 29 in the IPG by lead 24c. The stimulator also includes sensors 25 and 26, that are implanted on or in the stomach S, respectively, with leads 25a and 26a extending from the sensors 25 and 26 to the IPG 21. Sensor 26 is exposed to the inside of the stomach S while sensor 25 is attached to the outside of the stomach. Leads 22a, 23a, 24c, 25a and 26a are electrically coupled to the electronic circuitry 29 located in the CAN/IPG 21. FIG. 1B schematically illustrates an alternative system comprising lead 22c extending from multiple, individually-addressable electrodes 22b and electrically coupled to the electronic circuitry 29 located on the IPG 21.

A first exemplary sensor includes a core body temperature sensor for sensing temperature information. The potential for using temperature measurements to classify ingestion events is disclosed in Provisional U.S. Patent Application Ser. No. 61/166,636 filed on Apr. 3, 2009 (our Ref. No. 026458-001200US), U.S. patent application Ser. No. 12/754,435 (our Ref. No. 026458-1210US) filed on Apr. 5, 2010, and U.S. patent application Ser. No. 12/754,439 filed on Apr. 5, 2010 (our Ref. No. 026458-1220US), the contents of which are incorporated herein by reference. The sensor may be located on or extend from the IPG and/or the sensors may be located on or extend from a lead or other device. Alternatively or additionally, a sensor may be located separately on the stomach wall and/or a sensor may be otherwise positioned elsewhere within, coupled to or in communication with the patient. The sensors can be implanted for long term use of a month or more to generate signals correlating to energy expenditure of the body.

The second exemplary sensor comprises a heart rate sensor that collects information regarding HRV. The heart rate sensor may be located on an IPG and implanted in a patient; on a lead or other sensor body implanted separately from the IPG and coupled to the IPG, and/or may be part of an external sensor coupled to the patient such as a Holster monitor that is externally and non-invasively attached to a patient. In a preferred embodiment all or part of the CAN body acts as a reference electrode of high surface area in contact with tissue and fluids in the subcutaneous pocket. Another electrode, ideally with a significantly smaller surface area is on a lead that is in contact with the stomach wall, or in the subcutaneous space at a distance from the reference electrode integrated with the CAN body. An alternative embodiment comprises two electrodes on a lead attached to the stomach wall, where the distance between the electrodes is at least 2 cm, in a wide-spaced bipolar sensing configuration. The heart rate sensor generates signals correlating to heart rate information. These signals are collected and processed to determine the patient's HRV.

Other sensors may include food intake sensors and electrodes to measure gastric electrical activity (GEA). Desirably the electrodes would have a simple monopolar sensing configuration, with one or more electrodes sutured to the stomach wall, and a far field reference electrode. The same electrode could potentially be used for stimulation, sensing ECG, and GEA.

### Treatment System:

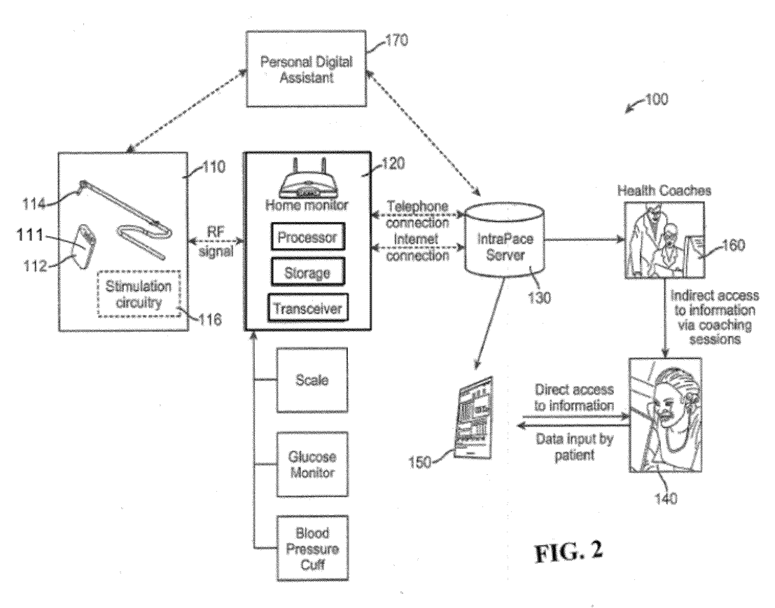

An example system 100 suitable for implementation in embodiments of the present invention is schematically illustrated in FIG. 2. System 100 comprises an implanted device 110 that communicates via a wireless transmitter disposed in an implant housing 112, such as an RF telemetry module. The wireless transmitter is located in an implantable pulse generator (IPG) 111. The implanted device 110 includes at least one sensor 114 and, optionally, stimulation circuitry 116 (typically disposed in-part in housing 112, and ideally also including an electrode disposed along a lead body coupling sensor 114 to housing 112) for providing therapeutic stimulation to the patient. A server 130 communicates with home monitor 120 via an internet or other telecommunication system so as to allow access to sensor-based data via a portal 150 and/or health coach workstation 160, thereby providing sensor-based feedback to a patient 140 (through direct presentation or display of the sensor-based information to the patient, and/or through a health-coach/patient relationship) and/or health care provider.

Each of implanted device 110, home monitor 120, server 130, health coach workstation 160, and portable patient device 170 will typically include associated data processing systems, with the overall feedback system 100 combining their data manipulation and communication capabilities into an overall data architecture. Generally, the data processing systems included in the discreet devices of the invention may include at least one processor. For implantable device 110, this will typically include circuitry implanted in the patient. Other devices of system 100 will include circuitry external to the patient. Such external processor circuitry may include one or more proprietary processor boards, and/or may make use of a general purpose desktop computer, notebook computer, handheld computer, smart phone, or the like. Further details regarding the hardware and software are disclosed in U.S. patent application Ser. No. 12/754,435 filed on Apr. 5, 2010 (our Ref. No. 026458-001210US), the entire contents of which are incorporated herein by reference.

Sensor 114 in FIG. 2 is coupled to the stomach so as to generate signals responsive to ingestion, with the sensor ideally comprising at least one temperature sensor for sensing temperature information from within the stomach. The sensors may be located on or extend from a housing of implanted device 110 and/or the sensors may be located on or extend from a lead or other device. Alternatively or additionally, a sensor may be located separately on the stomach wall and/or a sensor may be otherwise positioned elsewhere within, coupled to or in communication with the patient. At least one additional sensor comprising a heart rate sensor may be included, to measure patient HRV. The housing of implanted device 110 will typically contain a battery and circuitry of the implanted device, and may be similar to other known implantable stimulator housing structures used for heart pacemaker systems and the like. A suitable heart rate sensor may comprise an electrode or other sensor engaging the stomach wall so as to receive far field electric signals from the heart (with a device CAN or another electrode implanted subcutaneously acting as a reference). Optionally, such a heart rate sensor may employ the same electrode as used to stimulate stomach tissue to inhibit ingestion, though separate electrodes may alternatively be used. Other sensors that may be used to detect heart rate include acoustic sensors (that would measure heart sounds within the body), pressure sensors (positioned in the thoracic cavity would detect changes in pressure corresponding to the volume changes of the heart that occur with each heart beat). An accelerometer on the diaphragm may detect vibrations that correspond to the heart beat since the apex of the heart is very close to the diaphragm. In addition electrodes could be placed on the diaphragm (on the abdominal side) and detect the far field electrical signals corresponding to the heart beat. Many of these sensors could also be placed in the heart through a minimally invasive intravenous approach. Electrical, acoustic, or pressure heart signals, accelerometer signals, and/or other activity sensor signals may, like temperature, gastric electrical activity sensors, or other ingestion sensor signals, be processed and recorded using circuitry 116. Alternatively the heart rate sensor is included in a Holster monitor externally attached to the patient and in direct or indirect communication with the circuitry. Suitable sensors and implantable devices, as well as aspects of the other devices of system 100, may be described in (and/or may be modified from those described in) U.S. patent application Ser. No. 12/145,430, filed on Jun. 24, 2008 (our Ref. No. 026458-000610US) and U.S. patent application Ser. No. 10/950,345, filed on Sep. 23, 2004 (our Ref. No. 026458-000141US), both of which have previously been herein incorporated by reference. Processing of sensor signals so as to identify or classify ingestion events and/or patient activity level to be communicated by system 100 (which may occur partially or entirely in implanted device 110, home monitor 120, or server 130) may be more fully understood with reference to U.S. patent application Ser. No. 12/637,452, filed on Dec. 14, 2009 (our Ref. No. 026458-001110US), which was also previously incorporated herein by reference.

The server 130 contains a number of algorithms designed to evaluate the implanted device data logs in comparison with goals established by the patient and his or her health coaches 160. Based upon the results of the analysis, i.e. whether the goals have been met, coaching messages may be sent to the patient.

Both external and implanted memory of the devices of system 100 will often be used to store, in a tangible storage media, machine readable instructions or programming in the form of a computer executable code embodying instructions and/or data for implementing the steps described herein. The functions and methods described herein may be implemented with a wide variety of hardware, software, firmware, and/or the like as described in (and/or modified from those described in) U.S. patent application Ser. No. 12/754,435 filed on Apr. 5, 2010 (our Ref. No. 026458-001210US), the contents of which are incorporated herein by reference. Hence, the data processing functionality described herein (and/or the data manipulation method steps described herein) may be implemented largely or entirely within the implanted components, external to the patient, and locally, or remotely, though they may more commonly be distributed at least in part among some or all of the implanted, local, and/or remote data processing components.

As schematically depicted in FIG. 2, aspects of social networking systems 140, 150, 160, with sensor-based information that has been generated using signals from an implanted sensor may be made available to one or more members of a group. Such systems are disclosed in (and/or modified from those described in) U.S. patent application Ser. No. 12/754,435 filed on Apr. 5, 2010 (our Ref. No. 026458-001210US), the contents of which are incorporated herein by reference.

### Treatment Methods:

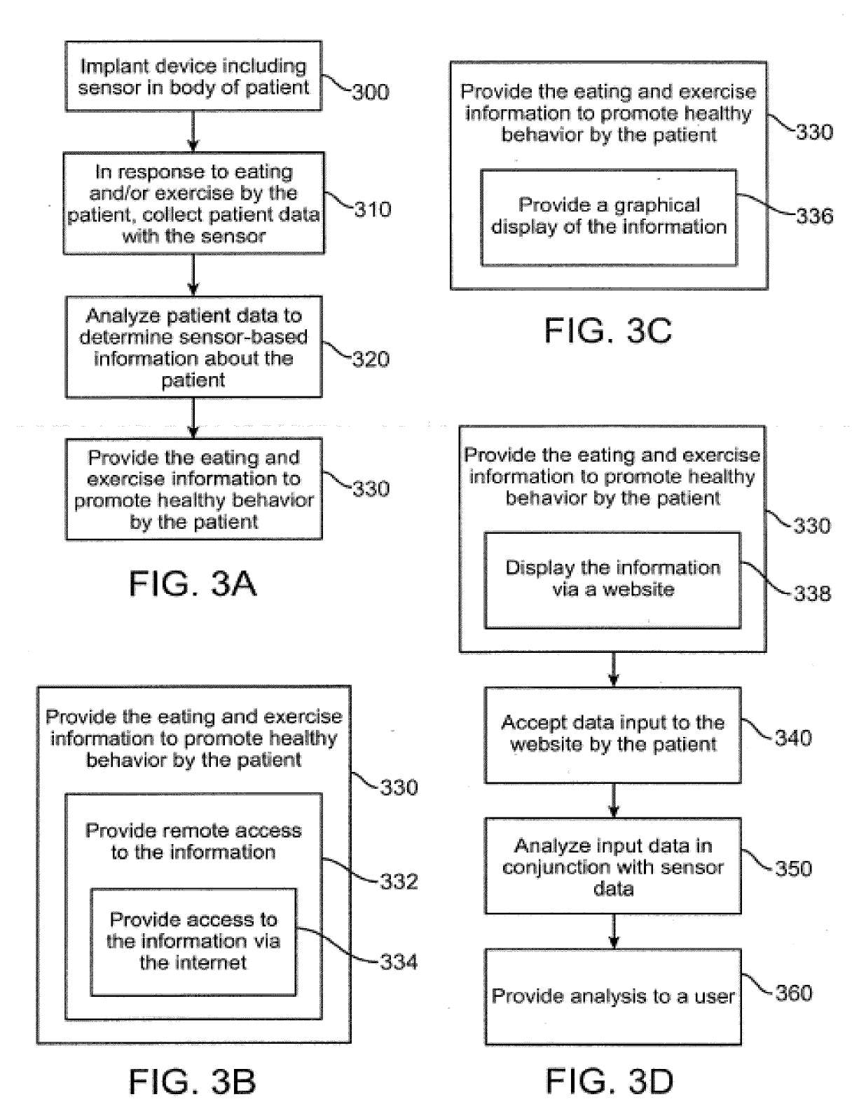

FIG. 3A illustrates a treatment method according to an embodiment of the present invention. Initially, a device including a sensor is implanted in the body of a patient 300. The device may be implanted in the stomach of the patient. Patient data is collected with the sensor in response to an ingestion event by the patient 310. The patient data is then analyzed to determine sensor-based information about the patient 320, including information based on the recorded HR and HRV and GEA of the patient. This diagnostic information could include heart health, sleep quality and sleep apnea diagnosis, stress level, fitness, and emotional state, and exercise diagnostics. The sensor-based information is provided to a user to promote the healthy behavior of the patient 330.

As shown in FIGS. 3B, step 330 may include providing remote access to the information 332, which may also include providing access to the information via the internet 334. In FIG. 3C, step 330 may include presenting a graphical display of the information 336. In some embodiments, such as illustrated in FIG. 3D, step 330 includes displaying the information via a website 338 and the method further includes accepting data input to the website by the patient 340 and analyzing the input data in conjunction with the sensor data 350. The resulting analysis is then provided to a user 360.

### Communication Methods:

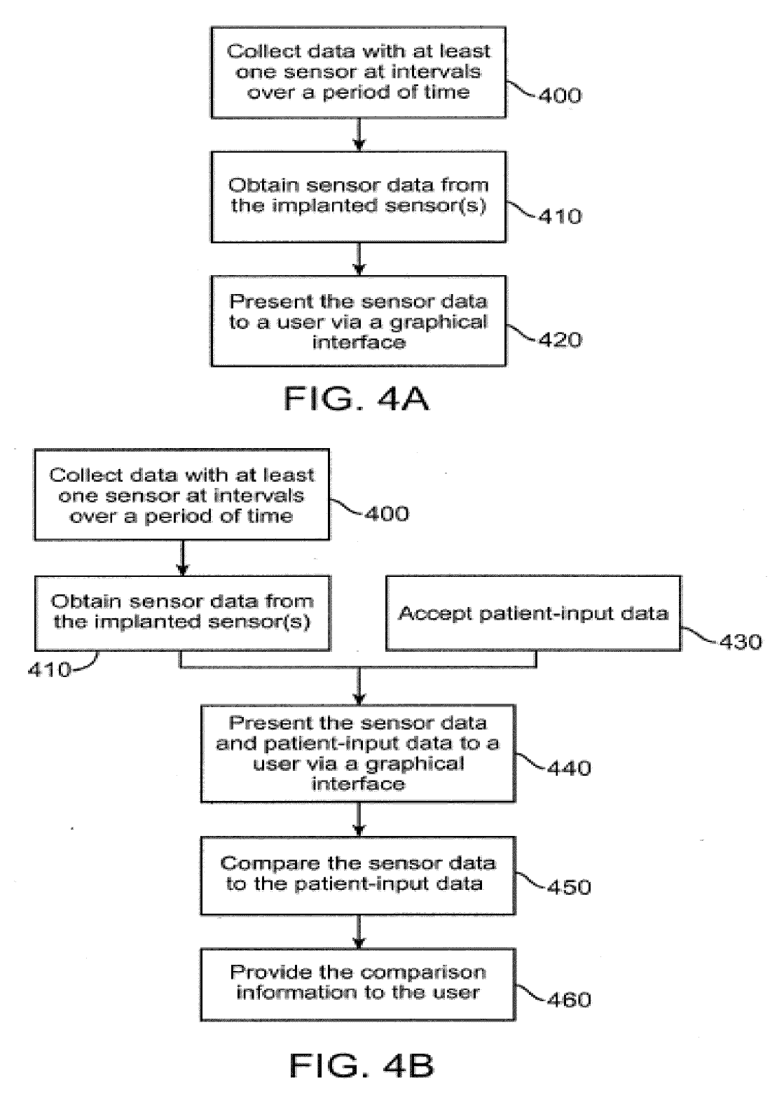

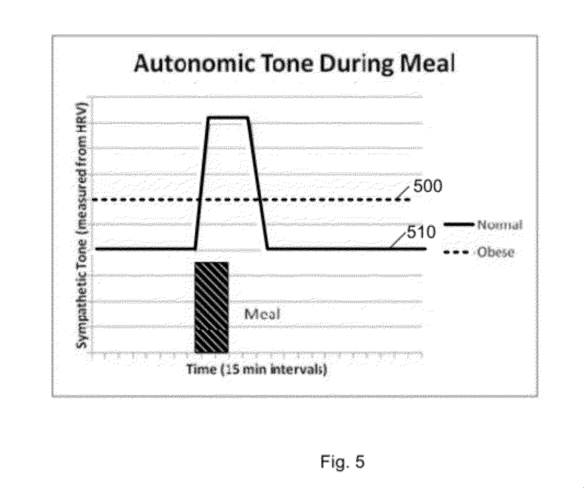

FIG. 4A illustrates a communication method according to an embodiment of the present invention. Data is collected by at least one implanted sensor at intervals over a period of time 400. The sensor data is obtained from the sensor(s) 410 and presented to a user via a graphical interface 420. The sensor data may also include information such as stress level, fitness, and emotional status which can be derived from sensors that provide information on autonomic tone such as HR and GEA. Referring to FIG. 4B, the method may include accepting patient-input data 430 and presenting both the sensor data and the patient-input data together 440. The sensor data and the patient-input data may also be compared 450 and the comparison information provided to the user 460. The sensor data may include ingestion and/or activity level information as further disclosed in U.S. patent application Ser. No. 12/754,435 filed on Apr. 5, 2010 (our Ref. No. 026458-001210US), the entire contents of which are incorporated herein by reference. FIG. 4C shows a sample display of a patient's caloric intake versus caloric output during a 24 hour period.

### Autonomic Balance Measurements and Control:

Various methods may be used to measure autonomic tone or balance. Lab work may be performed to measure arterial plasma concentrations or epinephrine or norepinephrine levels in urine or plasma. Another method involves measurement of tissue responsiveness to indirectly determine vagal activity. A characteristic of the autonomic nervous system is the non-uniformity of tissue responsivity, i.e. neural activity at one site does not guarantee similar activity at another tissue site. However, autonomic balance at the sinoatrial level can be determined by measurement of heart rate variation (HRV). Neural activation of cardiac tissue may correlate strongly with autonomic effects on energy metabolism elsewhere in the body. HRV may be used to indicate changes in the autonomic nervous system during a meal. HRV measurement is easily administered and may be performed non-invasively; for example, HRV can be measured by having a patient use a Holter monitor for a determined period of time, such as a few minutes or 24 hours. HRV may also be measured by an implanted heart rate monitor that is part of a more extensive therapy system. HRV can be measured with both long term and short term heart rate recordings. Physiological factors that affect HRV in an individual are gender, age, circadian rhythm, respiration, and body position.

The methodologies for calculating HRV can be divided into four main categories: time domain based, geometric methods, frequency domain (spectral analysis) based, and non-linear methods.

Time domain analysis methods that measure long term changes in heart rate variability include SDNN (standard deviation of NN intervals), SDANN (standard deviation of the average of NN intervals in all 5 minute segments of the entire recording), and SD (standard deviation of differences between adjacent NN intervals) may reflect day/night changes. Time domain methods that reflect short term changes in HRV include pNN50 (percent of difference between adjacent NN intervals that are greater than 50 ms), and RMSSD (root mean square of successive differences).

Geometric methods include triangular HRV index and the Poincare plot. The advantage of the geometric methods is that they are less affected by the quality of data (erroneous beats, artifacts, arrhythmias), but desirably at least 20 minutes of recording are available and analyzed.

The spectral analysis method may also be used for analyzing HRV. The power spectrum used for HRV analysis may be between 0 and 0.5 Hz. The high and low and very low frequency band can be analyzed with 5 to 10 minute recordings. The ultra low frequency band requires longer recordings. The spectral power in the low frequency band (0.04-0.15 Hz) can be used to represent sympathetic modulations and spectral power in the high frequency band (0.15-0.4 Hz) is generally used as a marker of vagal modulation. Also, a very low frequency band (0.003-0.04 Hz) may be a determinant of physical activity and sympathetic activity. The ultra low frequency (<0.003 Hz) band may reflect circadian rhythms. Non-linear methods may be very efficient at detecting abnormal changes in HRV, and may also be less sensitive to physiological changes such as body position and circadian rhythms.

Measures of HRV based on non-linear dynamics (NLD) may be divided into families, and a variety or prior HRV measures may be employed for (and/or modified for use in) the systems and methods described herein. One family is “fractal measures” which assess the self-affinity of heartbeat fluctuations over multiple time scales. These measures include Power-law correlation (scaling exponent β), Detrended fluctuation analysis (indices α1 and α2) and Multifractal analysis. Of these measures, Detrended fluctuation analysis with indices al may stand out as the best univariate predictor of mortality in patients with depressed left ventricular function after acute MI. This index may correlate with the spectral analysis measure LF/HF. A second family of NLD based measures is entropy measures, which assess the regularity/irregularity or randomness of heartbeat fluctuations. Two of these measures are Approximate Entropy (ApEn), and Sample Entropy (SampEn), both of which evaluate entropy on one time scale only and may be vulnerable to missed beats and artifacts. Multiscale entropy (MSE) assesses multiple time scales to measure a systems complexity. Another measure is Compression Entropy (CE) which quantifies the extent to which the data from heartbeat time series can be compressed, i.e. repetitive sequences occur. CE can be used to measure short term and long term changes and may correlate partly with SDNN and RMSSD. This measure may perform well for differentiating pathological HRV from healthy HRV in the case of cardiac diseases.

Poincare plot representation is another family of NLD based HRV measures, which assess the heartbeat dynamics based on a simplified phase space embedding. Poincare plots are a two dimensional graph with the RR(n) plotted against the next interval RR(n+1). Three indices are calculated from the Poincare plots, the standard deviation of the short term RR interval variability (minor axis of the cloud, SD1), the standard deviation of the long-term RR-interval variability (major axis of the cloud, SD2), and the axes ratio (SD1/SD2). SD1 may be able to differentiate the healthy subjects from all patients, in contrast to the time domain index RMSDD, which may be highly correlated with SD1.

Studies using non-linear measurements to access autonomic tone during meals or to access metabolism may be performed using approaches applied in any of a variety of prior studies directed to cardiac and other medical events, with appropriate modifications (optionally including both the time domain and frequency spectral domain). The use of nonlinear measurements to stratify risk in cardiac patients points to the benefit of using multivariate approaches, with non-linear dynamics based parameters in combination with standard linear parameters to improve the performance of HRV analysis. The limits of frequency analysis alone for determining the level of sympathetic and parasympathetic activation may also be incorporated into the analysis. This is because the different frequency bands are not a “pure” representation of either vagal or sympathetic activity. The HF components (˜0.4 Hz) are a result of sinus arrhythmia which is vagally based, but the effects of vagal control are seen in other frequency bands as well. There are also individual differences in the relationship between vagal activity and sinus arrhythmia. Thus the power in the high frequency (HF) band can be a highly inaccurate measure of vagal activity when used to compare groups of individuals such as obese vs. non-obese. Combining other approaches such as time based and NLD to a autonomic tone evaluation algorithm would help avoid the limitations of a solely frequency based approach.

### Screening Patients for Response to GES Therapy:

FIG. 5 is a schematic showing changes in autonomic tone during a meal for a normal and an obese person. Obese patients may have elevated baseline sympathetic tone compared to normal but have reduced or no elevation of sympathetic tone during meals. These obese patients may benefit from GES stimulation during meals to create the increase in sympathetic tone that leads to food thermogensis and increased energy expenditure during and following meals.

Sympathetic or vagal activation is organ specific and a rise in vagal input in the stomach will likely correspond to a reciprocal rise in sympathetic input to the heart. Thus, as a first step in the screening process the relationship between autonomic activation of the gastric system and the heart may be determined in order to use HRV with a desired confidence and/or efficacy in the treatment of obese patients. This relationship between autonomic activation of the gastric system and the heart may be established by performing baseline measurements with a patient at rest, after a stressful situation, and after ingestion of at least 500 kcal of food. The measurements may include both HRV measurement which measures autonomic balance at the heart level and spectral analysis of gastric electric activity which can measure changes in autonomic activation of the stomach. This baseline testing will establish expected baseline values for each patient for autonomic tone as well as detect the baseline levels of response to certain events such as stress and food ingestion. The expected response to a stressful event is an increase in sympathetic tone at both the cardiac and digestive system level. On the other hand meal ingestion causes an activation of vagal efferent nerves to the tissues involved in digestion, and a reduction in vagal tone in the cardiac tissue (except for possibly the first 5 minutes following a meal) as shown in studies using HRV to monitor autonomic tone during a meal. Spectral analysis methods may be applied to the HRV data of a patient to identify a reduction in the high frequency (HF) component in the first hour following a meal, and an increase in the LF/HF ratio (LF being low frequency), these measures signifying a vagal withdrawal.

Obese patients will also be screened by non-invasive GES with endoscopically-placed leads to measure their autonomic response using HRV. The change in autonomic tone in magnitude and direction of the change in autonomic tone will be used to determine if the patient is actually responsive to GES therapy. Also a high sympathetic tone at baseline measured with HRV may indicate the presence of metabolic syndrome which could be factored into the patient screening process. Gastric electrical activation (GEA) can also be measured non-invasively with surface electrodes placed on the stomach and feedback from this measurement may be used in the screening process. Typically three disposable electrodes will be used, one placed on the abdominal midline just above the umbilicus, and a second approximately 6 cm to the left and 3 cm superior to the midline electrode, and a reference electrode positioned approximately 10 cm to the right of the midline and 3 cm above the umbilicus. The EGG signal should then be passed through a bioamplifier and digitized. Spectral analysis of the signal can be used to determine changes in autonomic tone at the level of the digestive system, where more power in the normal bandwidth may be indicative of increased vagal tone, and more power in the tachyarrhythmic bandwidth was indicative of increased sympathetic tone. The normal bandwidth may be set at 2.5-3.75 cpm, and the gastric tachyarrhythmic bandwidth may be 4-9.75 cpm.

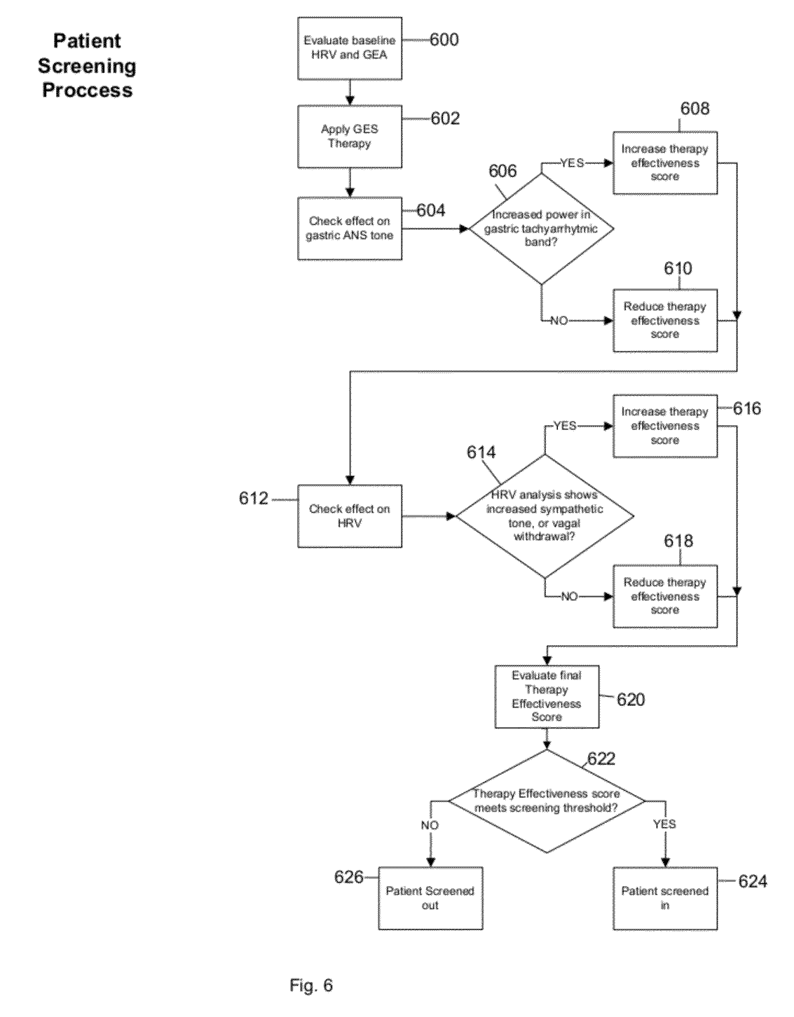

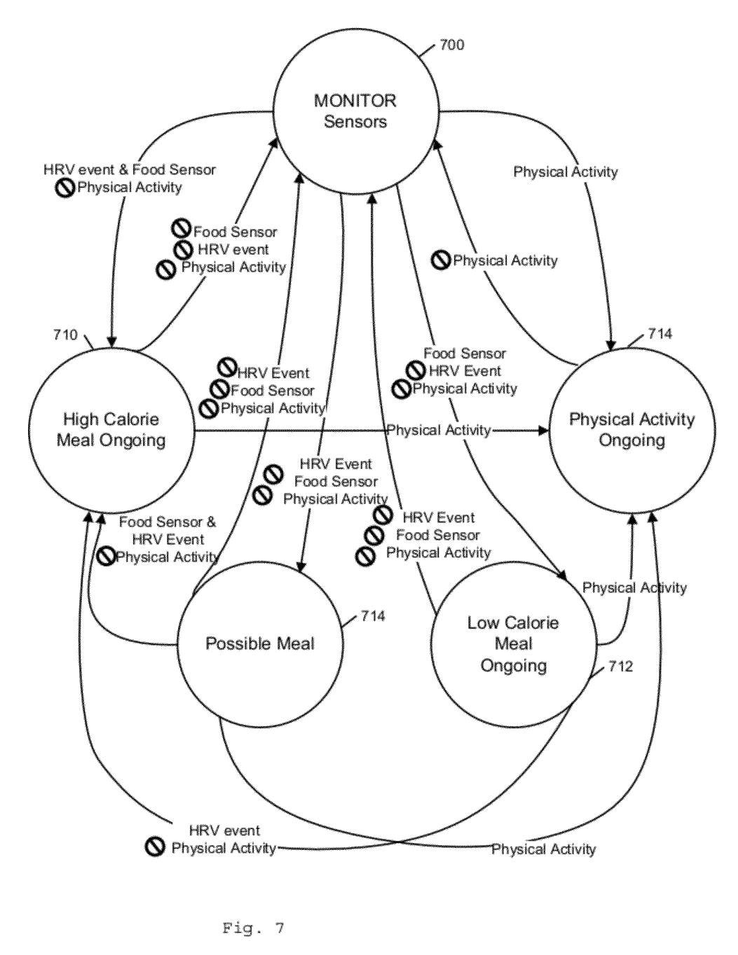

FIG. 6 shows how measurement of HRV and gastric ANS tone can be used to determine if a patient is a good candidate for GES therapy. First the baseline HRV and GEA are evaluated 600, then GES therapy can be applied for a certain time window (for example 5 min) 602. The effect of the GES on gastric ANS tone can then be determined through spectral analysis as discussed above 604. For example, the ratio of the spectral power in the tachyarrhythmic bandwidth over the total spectral power can be evaluated before and after GES 606. An increased ratio is indicative of increased sympathetic tone at the gastric level, and indicates higher therapy effectiveness 608, if not the therapy effectiveness is reduced 610. The effect of GES on HRV can be evaluated using methods such as discussed above 612. For example the RMSSD or the Poincare plot (index SD1) could be used to determine if vagal withdrawal occurs 614. The occurrence of vagal withdrawal may indicate improved therapy effectiveness 616, and absence of it may indicate reduced therapy effectiveness 620. Finally the therapy effectiveness score is evaluated and compared to a pre-determined screening threshold 622. If patients meet this screening threshold, then GES therapy is more likely to be effective for them 624 than if the screening threshold is not met and the patient is screened out 626.

Furthermore, the patient's response to GES therapy will also be used to determine the optimum placement of the one or more GES electrodes. Once optimal locations for GES therapy will be determined, markers will be placed endoscopically to pinpoint these locations for the actual implantation of the GES system. Probes for stimulating candidate treatment sites may include the endoscopic probes (and/or be modified from the probes) described in US Patent Publication No. 2009/0149910 with reference to FIGS. 35A and 35B, but with HRV analysis of the patient response (optionally in combination with the disclosed electromyographic (EMG) analysis). A wide variety of alternative evaluation probes might also be employed.

### Meal Detection

HRV may be used to detect the onset of the cephalic state since vagal activation in the gastric system occurs starting with the cephalic state, which begins secretion of gastric juices (which can also be detected with HRV changes). Implanted GES devices and systems capable of monitoring HRV may differentiate these changes in the cephalic state and start therapy to pre-empt eating and increase the impact of the therapy.

In one embodiment, a number of HRV paramaters such as RMSSD, LF/HF, and Entropy will be monitored during the timespan of 24 hours to one week. The HRV parameters will act as input signals to a learning algorithm that will store these signals for a time window, desirably for 10-30 minutes, around a meal event that is detected by a food sensor. During the learning period of 24 hours to one week, a database of these HRV signal windows will be created. These HRV signal windows will then be processed to create a “template(s)” that will represent the average change that these one or more signals undergo during a meal event. The template(s) will include required limits for pattern matching. If more than one HRV parameter is used, then the change in each of these parameters must match its respective template.

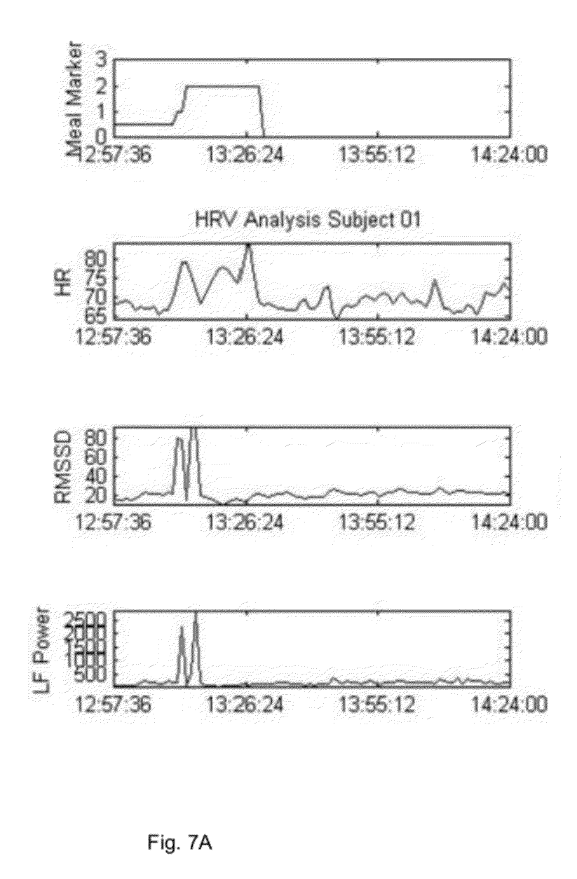

FIG. 7A shows the changes that can occur in a number of different HRV signals during a meal (HR, RMSSD, LF Power). The top plot is the meal marker, which is high for the duration of the meal. These signals can represent multiple templates. The pattern matching may require that each time sample of the HRV signal be within a range of the template value, or the template can be represented by a set of parameters describing the signal, such as amplitude, slope, area under the curve, etc.

As shown in FIG. 7, HRV may be used for meal detection. A baseline is established using diurnal variations based on HRV metrics taken over a period of several days. Event based changes in HRV are determined and then used to establish thresholds. Current baseline autonomic tone from HRV taken over a few minutes is compared with diurnal record to detect a potential HRV event. Significant HRV changes may occur with ingestion of food of 400 calories or more. Thus the significant HRV changes will be used to signal significant ingestion events or caloric intakes, especially in the context of verifying the accuracy of the meal input information in the patients' diaries.

For this algorithm, the processor employs a food sensor event to determine if a meal is taking place, and uses the HRV event to determine if it is low or high calorie. During the “Monitor Sensors” state 700, the HRV sensor, the activity sensor/s, and the food detection sensor/s are monitored. If a food sensor event and an HRV event occur with no activity, the processor enters “High Calorie Meal Ongoing” state 710. If a food sensor event occurs without any HRV or physical activity events, then the “Low Calorie Meal Ongoing” state 712 is entered. If an HRV event occurs with no physical activity and no food sensor events, then “Possible Meal” state 714 is entered. A physical activity event overrides all meal detects, and leads to the end of any ongoing meals states—High Calorie Meal, Low Calorie Meal, or Possible Meal—and the processor enters and ‘Physical Activity Ongoing” state 716, until the activity detection ends, and the processor returns to the “Monitor Sensors” state 700. If the processor is in “Low Calorie Meal Ongoing” state 712 and an HRV event occurs with no physical activity then the “High Calorie Meal Ongoing” state 710 is entered. Once the food sensor event ends, the processor returns to “Monitor Sensors” state 700. If the processor is in “High Calorie Meal Ongoing” state 710 and the HRV event ends, then the machine returned to “Monitor Sensors” state 700.

The Table below shows a more complete implementation of the possible events and the next action(s) to be followed, dependent upon the current state.

| | | Physical | Food | |
|---|---|---|---|---|
| | HRV | Activity | Sensor | Next Action |
| Current State | event | Event | Event | (Go to . . . State) |
| Monitor Sensors | + | Ø | Ø | Possible Meal |
| Monitor Sensors | Ø | Ø | + | Low Calorie Meal |
| | | | | Ongoing |
| Monitor Sensors | + | Ø | + | High Calorie Meal |
| | | | | Ongoing |
| Monitor Sensors | Ø or + | + | Ø or + | Physical Activity |
| | | | | Ongoing |
| Physical Activity | Ø or + | Ø | Ø or + | Monitor Sensors |
| Ongoing | | | | (& Record End of |
| | | | | Activity) |
| Low Calorie Meal | + | Ø | Ø or + | High Calorie Meal |
| Ongoing | | | | Ongoing |
| Low Calorie Meal | Ø or + | + | Ø or + | Physical Activity |
| Ongoing | | | | Ongoing (& Record |
| | | | | End of Meal) |
| Low Calorie Meal | Ø | Ø | Ø | Monitor Sensors |
| Ongoing | | | | (& Record End of |
| | | | | Meal) |
| Possible Meal | + | Ø | + | High Calorie Meal |
| | | | | Ongoing |
| Possible Meal | Ø or + | + | Ø or + | Physical Activity |
| | | | | Ongoing |
| Possible Meal | Ø | Ø | Ø | Monitor Sensors |
| High Calorie Meal | Ø | Ø | Ø or + | Monitor Sensors |
| Ongoing | | | | (& Record End of |
| | | | | Meal) |
| High Calorie Meal | Ø or + | + | Ø or + | Physical Activity |
| Ongoing | | | | Ongoing (& Record |
| | | | | End of Meal) |

In another embodiment a meal may be detected using only HRV events, given that the number of false detects using the HRV event based meal detection is sufficiently low.

HRV may also be used to detect the end of a meal and satiety. A decrease in parasympathetic input at the cardiac level may occur in the first 30 minute period after food ingestion. Slight increase of high frequency (HF) amplitude or vagal tone may take place in the first 5 minutes after a test meal.

A decrease in RMSSD levels may indicate higher sympathetic tone and decrease in nocturnal HRV in chronic overeaters with raised glucose levels. HRV could also be used for detecting eating patterns and chronic glucose imbalance in obese subjects.

### Therapy Titration

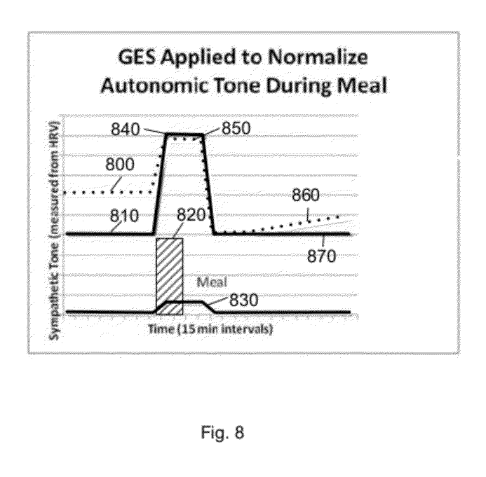

HRV analysis will be used for GES therapy titration. A patient's initial HRV rate will be determined before actual GES stimulation and then will be continuously monitored during the GES therapy follow-up. As shown in FIG. 8 the goal is to set a patient's HRV level between certain parameters, given the patient's baseline HRV by activating the sympathetic tone and without causing excessive stress, which may be both detrimental to the obese patient's health overall and may actually cause the patient to start overeating. The advantage of using HRV data for GES therapy titration is that the effectiveness of the GES therapy is measured using objective criteria, rather than a patient's subjective evaluation of pain and other sensations.

FIG. 8 shows the sympathetic tone for an obese patient 800 and a normal person 810. During a meal 820, stimulation is turned on 830. The obese patient's sympathetic tone is increased 840 to resemble the sympathetic tone of a normal person 850. When the stimulation is turned off there is a possible reduction in sympathetic tone and/or increase in vagal tone that helps normalize the baseline autonomic tone in the obese; the stimulation causes the obese patient's baseline HRV to drop 860 and to come close to the baseline HRV level of a normal person 870.

As obese patients lose weight, their HRV level will often increase; thus the parameters showing effectiveness of the GES therapy may be re-set on a regular basis, optionally at least once each month, once each week or even as often as once during each 24-hour period. Thus, in one embodiment of the invention, the HRV baseline level will be re-determined at a given time during the night when the obese patient is asleep and then the level of GES therapy will be adjusted accordingly. A preferred HRV methodology for real-time or near real-time algorithm would respond to short term changes in HRV. An example would be the RMSSD time domain method. In this case the RMSSD could be calculated on a sliding two minute window of R-R intervals. At any desired point in time the HRV could be determined from the previous 2 minute window. This running calculation of RMSSD may appear as shown in FIG. 8 during a meal. A detection algorithm could be applied to this real-time RMSSD output that includes appropriate qualifiers for detecting a meal. These qualifiers could be used in a support vector algorithm, or they could be part of a decision tree approach, where the real time RMSSD signal may have to meet one or more qualifications in order for a meal to be detected. These qualifiers may include amplitude, slope, area under the curve, variability, etc. The same set of qualifiers (or optionally different qualifiers) may be used to detect the end of the meal, or satiety level. In addition, this signal may be used to determine if there are abnormal autonomic system responses to a meal.

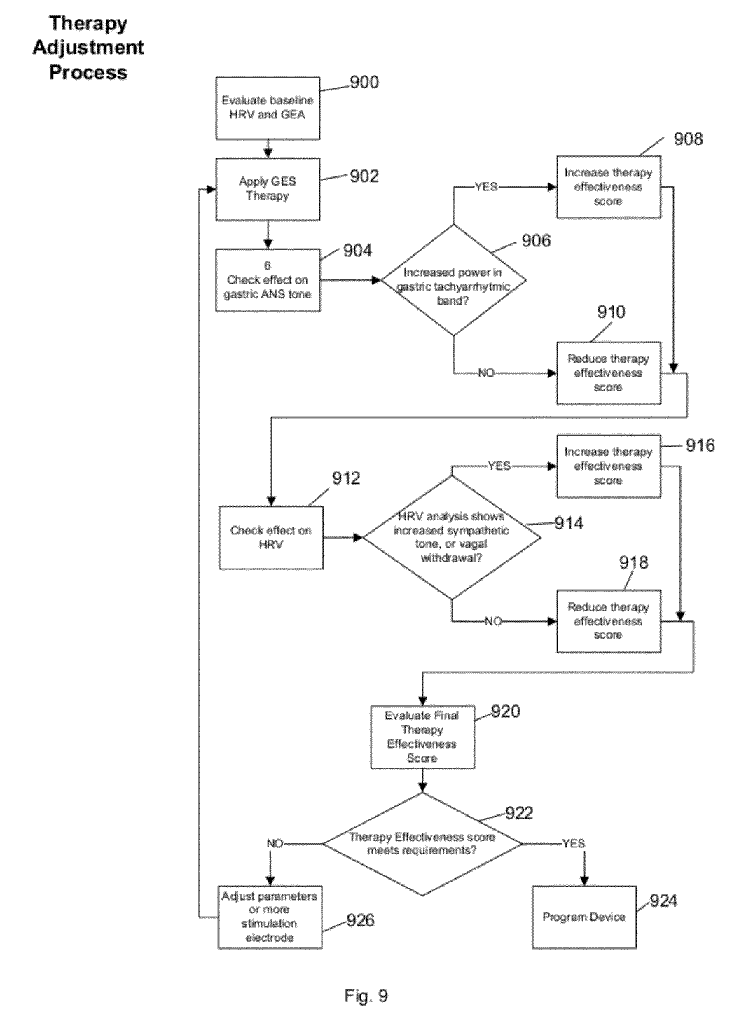

If the implanted system includes a GEA signal then the therapy titration algorithm could include analysis of gastric response in the adjustment of therapy parameters. FIG. 9 is a flow chart illustrating how these two parameters may be used to determine if therapy should be adjusted. This algorithm may be implemented in the software run on the external instrumentation used to program the implanted device. Telemetry could be used so the external instrument could obtain raw signals from the ECG and GEA sensors, optionally in real-time. Or the algorithm could be implemented in device firmware for automatic therapy adaptation. The evaluation and therapy adaptation could be programmed to occur at certain intervals following device implantation and therapy activation.

FIG. 9 shows a method of GES therapy titration. First the baseline HRV and GEA signals will be evaluated 900. Next a therapy will be triggered by the external instrument 902 (or by the device if automatic therapy adaptation is implemented), and the effect on gastric ANS tone is determined 904. Spectral analysis methods or other analysis methods (such as nonlinear dynamics based methods) will be used to determine the level of tachyarrhythmia in the stomach compared to baseline and therefore the gastric ANS tone 906. An increase in tachyarrhythmia or a reduction in normal rhythm indicates increased therapy effectiveness 908, while increased normal rhythm may indicate reduced therapy effectiveness 910. Next HRV will be analyzed 912 (again during GES therapy), and compared to baseline 914. Vagal withdrawal will indicate more effective therapy when delivered during a meal 916, while lack of vagal withdrawal or vagal tone increase would not 918. The final therapy effectiveness score would then be evaluated 920, relative to the desired effectiveness score 922. The desired effective score may be the same across the patient population or individualized for a patient based on initial testing and response. If the desired effectiveness score is not met, then the therapy parameters will be adjusted and the test repeated 926, otherwise the device will be programmed with the current parameters 924.

While exemplary embodiments have been described in some detail for clarity of understanding and by way of example, a variety of adaptations, modifications, and changes will be obvious to those of skill in the art. Hence, the scope of the present invention is limited solely by the appended claims.

What is claimed is:

1. A system for performing diagnostic or therapeutic functions for a patient having an ingestion-related disorder, the system comprising:
a sensor configured to collect data from the patient;
a treatment applicator or display; and
a processor coupling the sensor to the treatment applicator or display, the processor configured to:
identify a baseline autonomic nervous system balance in response to the sensed data;
identify an excursion of the autonomic nervous system balance from the baseline, the excursion associated with a discrete ingestion or stimulation event;
evaluate the event using the baseline autonomic system balance and
to transmit command signals to the treatment applicator or display in response to the evaluation of the event so as to promote ingestion modification by the patient.

2. The system of claim 1, wherein the sensor comprises a heart beat signal sensor and the autonomic nervous system balance baseline and excursion are determined by generating heart rate variability information during the event.

3. The system of claim 2, wherein the heart beat signal sensor is configured to be implanted into a body of the patient and the treatment applicator is configured to be implanted into the body coupled to a tissue of the gastrointestinal tract or associated nerves of the patient to stimulate the tissue so as to inhibit unhealthy ingestion into the patient in response to the heart rate variability information.

4. The system of claim 3, wherein the treatment applicator, when implanted, stimulates the tissue in response to the command signals during the event.

5. The system of claim 4, wherein the processor is configured to identify the event as an unhealthy ingestion event using at least the heart rate variability information.

6. The system of claim 1, wherein the event comprises a stimulation event, wherein the processor is configured to alter stimulation applied by the treatment applicator in response to the heart rate variability information, the evaluation of the event comprising an evaluation of effectiveness of the stimulation at inducing a desired temporary excursion from the baseline autonomic nervous system balance during the stimulation.

7. The system of claim 6, wherein the processor is configured to initiate the stimulation event in response to ingestion by the patient, the event also comprising an ingestion event.

8. The system of claim 6, wherein the display shows patient selection information in response to the command signals, the stimulation applicator comprising a patient evaluation probe and the stimulation event comprising a patient evaluation stimulation event.

9. A system for performing diagnostic or therapeutic functions for a patient having an ingestion-related disorder, the system comprising:
a sensor configured to collect data from a tissue within the patient;
a display; and
a processor coupling the sensor to the display, the processor configured to determine autonomic nervous system balance information in response to the sensed data and to transmit command signals to the display so as to generate an output; and
a gastric stimulator system having a stimulation surface coupleable to a tissue of a gastrointestinal tract or associated nerves in response to the determined autonomic nervous system balance information.

10. The system of claim 9, wherein the sensor comprises one of a heart rate sensor and a gastic electrical activity sensor.

11. The system of claim 9, the patient comprising an obese patient, wherein the processor is configured to receive a first set of the data associated with a first portion of a time span taken before an ingestion event while the obese patient is resting, a second set of the data associated with a second portion of the time span taken while the obese patient is exercising, and a third set of the data associated with a third portion of the time span during an ingestion event of the obese patient; and
wherein the processor is configured to calculate an overall autonomic nervous system balance of the body for the time span.

12. The system of claim 11, wherein the output by the display indicates whether the gastric electrical stimulation therapy would be an effective treatment of obesity in the patient.

13. The system of claim 11, wherein the processor is configured to compare the overall autonomic nervous system balance of the body to an autonomic nervous system during another time span so as to detect stress and/or activity during the other time span, the overall autonomic nervous system balance comprising a baseline.

14. The system of claim 9, further comprising an endoscopic probe having a test stimulation surface for stimulating a candidate location on a wall of a stomach of an obese patient;
wherein the output of the display indicates whether a gastric electrical stimulation therapy with the stimulation surface at the candidate location of the lead will be effective.

15. A method for treating a patient having an ingestion-related disorder, the method comprising:
collecting heart rate variability data from the patient;
identifying an unhealthy or weight gain promoting ingestion event in response to the heart rate variability data;
promoting ingestion modification by the patient in response to the identification of the event.

16. A method for treating a patient having an ingestion-related disorder, the method comprising:
collecting heart rate variability data from the patient;
identifying a termination of an ingestion event in response to the heart rate variability data;
promoting ingestion modification by the patient in response to the identification of the event.

17. A method for treating a patient having an ingestion-related disorder, the method comprising:
collecting heart rate variability data from the patient;
identifying a pre-eating stage of an ingestion event in response to the heart rate variability data;
promoting ingestion modification by the patient in response to the identification of the event.

18. A method for selecting a patient for an ingestion-behavior modification therapy, the method comprising:
stimulating a tissue of the gastrointestinal tract or associated nerves of the patient;
collecting heart rate variability data from the patient while stimulation of the tissue induces a discrete change in the heart rate variability data ;and
screening the patient for implantation of an ingestion-behavior modification implant in response to the change in the heart rate variability data.

19. A method for controlling a therapy for an obese patient, the method comprising:
stimulating a tissue of the gastrointestinal tract or associated nerves of the patient;
collecting heart rate variability data from the patient while stimulation of the tissue induces a discrete change in the heart rate variability data ;and
altering the stimulation of the tissue in response to the change in the heart rate variability data, the heart rate variability data providing a stimulation effectiveness feedback signal.

20. A method for selecting a patient for an ingestion-behavior modification therapy, the method comprising:
stimulating a tissue of the gastrointestinal tract or associated nerves of the patient;
collecting autonomic nervous system balance data from the patient while stimulation of the tissue induces a discrete change in the autonomic nervous system balance data; and
screening the patient for implantation of an ingestion-behavior modification implant in response to the change in the autonomic nervous system balance data.

21. A method for controlling a therapy for an obese patient, the method comprising:
stimulating a tissue of the gastrointestinal tract or associated nerves of the patient;
collecting autonomic nervous system balance data from the patient while stimulation of the tissue induces a discrete change in the autonomic nervous system balance data; and
altering the stimulation of the tissue in response to the change in the autonomic nervous system balance data, the heart rate variability data providing a stimulation effectiveness feedback signal.
